SEQUENCE LISTING (10 sequences)

SEQ ID NO: 1 - protein, 5 aa, artificial sequence
  beta-sheet binding peptide
RVVIA

SEQ ID NO: 2 - protein, 5 aa, artificial sequence
  beta-sheet binding peptide
KLVFF

SEQ ID NO: 3 - protein, 5 aa, artificial sequence
  beta-sheet binding peptide
LPFFD

SEQ ID NO: 4 - protein, 4 aa, artificial sequence
  beta-sheet binding peptide
IIGL

SEQ ID NO: 5 - protein, 5 aa, artificial sequence
  beta-sheet binding peptide
RIIGL

SEQ ID NO: 6 - protein, 5 aa, artificial sequence
  beta-sheet binding peptide
GVVIA

SEQ ID NO: 7 - protein, 6 aa, artificial sequence
  beta-sheet binding peptide
RRAFFV

SEQ ID NO: 8 - protein, 4 aa, artificial sequence
  beta-sheet binding peptide
IIGL

SEQ ID NO: 9 - protein, 5 aa, artificial sequence
  beta-sheet binding peptide
RIIGL

SEQ ID NO: 10 - protein, 6 aa, artificial sequence
  beta-sheet binding peptide
RRAFFV

(19) United States
(12) Patent Application Publication
(10) Pub. No.: US 2006/0094071 A1
(43) Pub. Date: May 4, 2006

(54) Method for enriching and tracking pathologic modified prions-proteins(prpsc)

(76) Inventors: Claudia Engenann, Leipzig (DE); Katja Hoeschler, Cambridge (GB); Jorg Lehmann, Borsdorf (DE); Jorg Gabert, Leipzig (DE); Ulrike Krummrei, Leipzig (DE)

Correspondence Address: FULBRIGHT & JAWORSKI L.L.P., 600 CONGRESS AVE., SUITE 2400, AUSTIN, TX, 78701, US

(21) Appl. No.: 10/520,386

(22) PCT Filed: Jul. 4, 2003
(86) PCT No.: PCT/DE03/02249

§ 371 Date: Sep. 8, 2005

(30) Foreign Application Priority Data
Jul. 4, 2002 (DE) 10230141.7

(51) Int. Cl. G01N 33/537; G01N 33/53; G01N 33/543

(52) U.S. Cl. 435/7.92

(57) ABSTRACT

The invention relates to a method for enriching and tracking pathologic modified prions-proteins (PrP^{sc}) of living organisms.

The present invention relates to a method for the detection of pathologically-altered prion proteins of living organisms.

Transmissible Spongiform Encephalopathies (TSE) are infectious, always fatal, degenerative diseases of the central nervous system. The histopathological changes in the brain occurring in these diseases are associated with the accumulation of pathologically-altered prion protein (PrP^{Sc}), a conformer of the naturally-occurring cellular prion protein (PrP^{C}). The prion replication, which occurs during the course of the disease, takes place as a result of a direct interaction between PrP^{Sc} and PrP^{C}, wherein the conformation of the pathological PrP^{Sc} is forced onto the normal PrP^{C}. By contrast with PrP^{C}, PrP^{Sc} is characterised by an increased content of β-pleated-sheets and a high resistance to proteases (e.g. proteinase K).

This resistance of PrP^{Sc} is currently utilised in the context of in-vitro diagnostics for the detection of Bovine Spongiform Encephalopathy (BSE). The principle of the current test system consists in homogenising tissue parts from the brain stem (obex region) and treating them with proteinase K. The protease treatment completely breaks down the normal PrP^{C}, but the PrP^{Sc} from BSE-infected animals is only shortened by a few amino acids, which reduces the relative molecular mass from 33-35 kDa to 27-30 kDa. Following this, the remaining PrP is visualised using the Western-Blot or ELISA techniques with the assistance of monoclonal antibodies.

The crucial disadvantage of this test system is its low sensitivity. The concentration of PrP^{Sc} in BSE-infected cattle is only sufficiently high for existing test systems in the central nervous system (CNS), and accordingly, diagnosis has hitherto been limited to post-mortem tests and relies on an incubation period of the infected organism of at least 18 months to several years.

In addition to the detection of PrP^{Sc} described above, there are two other methods for the diagnosis of TSEs: a histopathological method for detection of the typical spongiform changes in the CNS and a bioassay, which demonstrates the infectiousness of specimens in the mouse model. Both of these methods also have crucial disadvantages. The histopathological method is not suitable for preclinical diagnostics, because the structural changes in the brain only occur at a late stage of incubation, shortly before the clinical phase. Moreover, with this method also, the diagnosis is made post mortem, because the necessary brain matter cannot be obtained from the living organism. In theory, the bioassay is capable of detecting a single infectious unit, but this method requires at least several months or even years.

To perform the diagnosis as quickly as possible after a potential infection and/or in an organism, which is still living, the sensitivity of the previous detection method must be substantially increased, and detection must be made possible in tissue/body fluids other than the CNS.

A binding of a pathological PrP^{Sc} to human serum proteins such as plamsinogen has been described by Fischer et al. [Fischer, MB; Roeckl, C; Parizek, P; Schwarz, HP; Aguzzi, A (2000); “Binding of disease-associated prion proteins to plasminogen”. Nature, 408: 479-483]. The binding of the prion proteins to plasminogen is dependent upon the conformation of the protein, because PrP^{C} cannot be bound. Fibrinogen is also capable of binding PrP^{Sc} but not in the absence of Ca²⁺ ions.

According to WO 02/00713, the specific binding of PrP^{Sc} to human plasminogen is used for the isolation of PrP^{Sc} from CNS material. For this purpose, human plasminogen is immobilised on magnetic particles. However, this method is only suitable for the detection of PrP^{Sc} in body fluids and tissues, which contain no plasminogen; it is not suitable, for example, for the detection of PrP^{Sc} in serum. Other disadvantages of this method are its costliness and the limited protein-binding capacity of the plasminogen-loaded magnetic particle.

The present invention is therefore based on the object of providing a method, which achieves increased sensitivity and allows a diagnosis of TSE in living organisms.

This object is achieved by the subject matter defined in the patent claims.

The invention is explained with reference to the following diagrams.

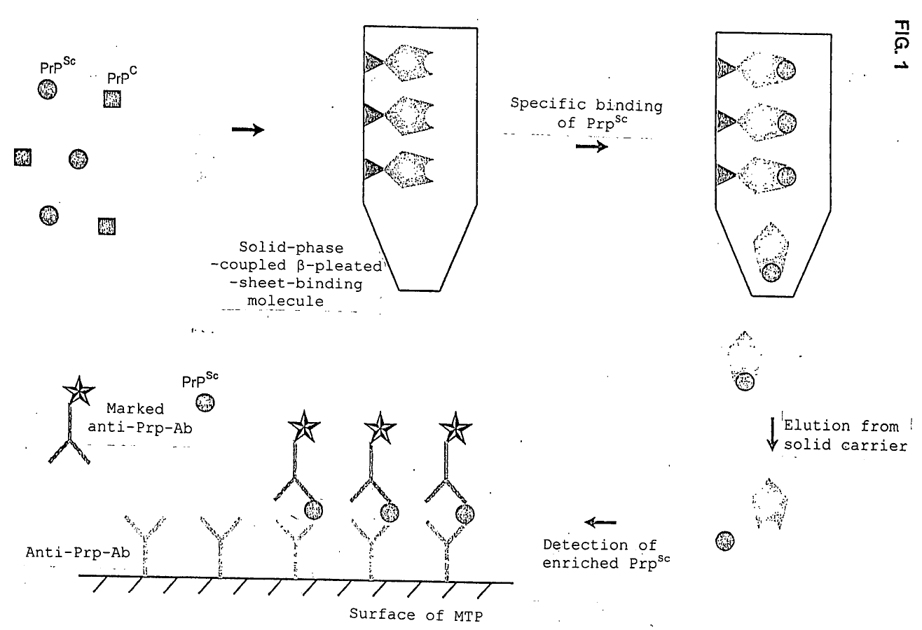

FIG. 1 shows diagrammatically the principle of the method according to the invention for the enrichment and detection of PrP^{Sc} using solid-phase-coupled β-pleated-sheet-binding molecules.

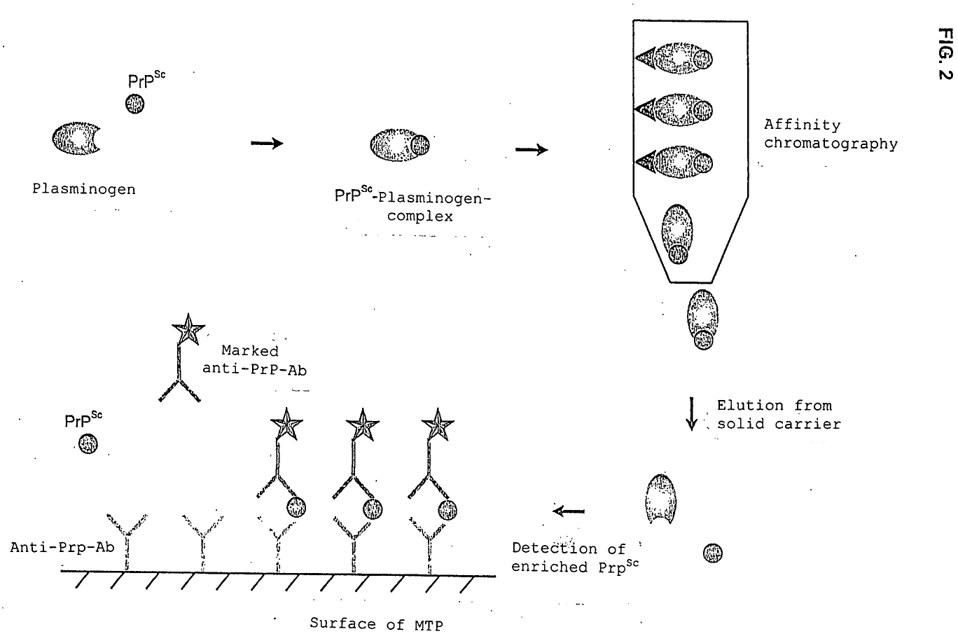

FIG. 2 shows schematically the structure of the PrP^{Sc} binding assay in the microtitre-plate format (MTP) (Example 1). Various β-sheet-breaker (BSB) peptides were immobilised on a carrier as potential β-pleated-sheet-binding-molecules and PrP^{Sc} catchers; after incubation with the specimen material, the bound PrP^{Sc} was visualised using monoclonal anti-PrP-antibodies.

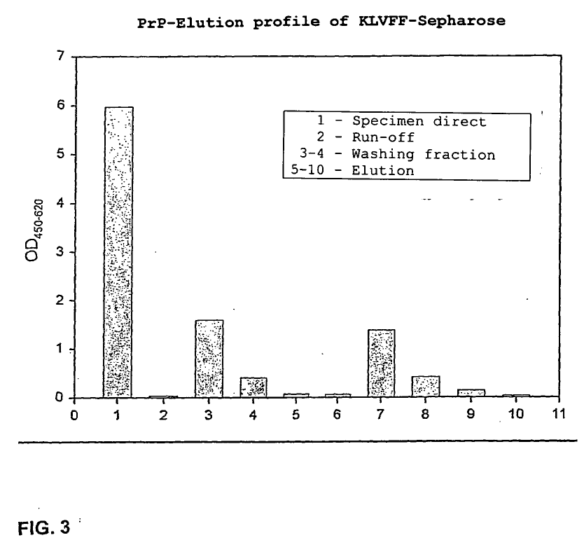

FIG. 3 shows an elution profile of the PrP^{Sc} content in the individual fractions after the binding of PrP^{Sc} from brain homogenate of BSE-positive cattle to KLVFF-sepharose. The quantity of PrP^{Sc} contained in the fractions was determined in a semi-quantitative manner using the Platelia® BSE Detection Kit and is expressed in Optical Density [OD] values.

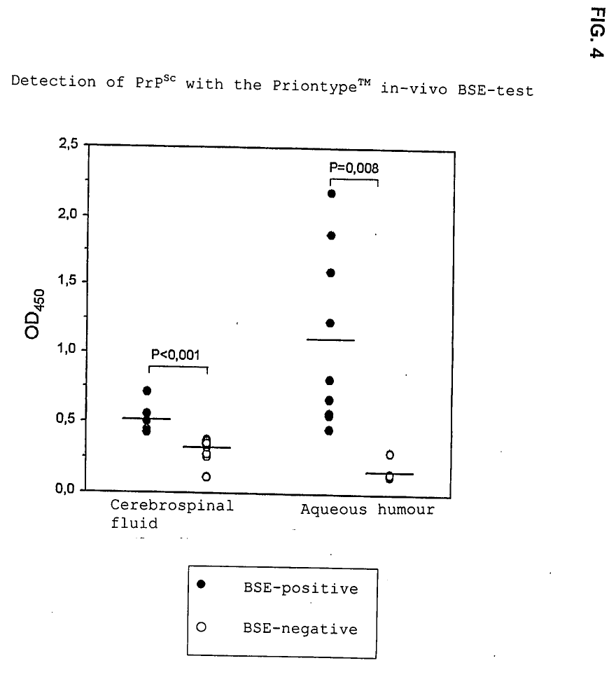

FIG. 4 shows an evaluation of the detection of PrP^{Sc} in aqueous humour and cerebrospinal-fluid specimens from BSE-positive cattle. In this example, the peptide KLVFF was used in the ELISA format as a catcher molecule for PrP^{Sc}. The detection was carried out with a monoclonal antibody V5B2 (r-Biopharm, Darmstadt) directed against PrP^{Sc} and a polyclonal peroxidase-conjugated goat-anti-mouse-IgG antibody.

The term “β-pleated-sheet-binding molecule”, as used in the present context, describes an organic molecule, which is capable, because of its three-dimensional structure and/or its physical properties, of interaction with β-pleated-sheet structures in proteins, e.g. in pathologically-altered monomer/oligomer prion proteins, and of binding them on the basis of this interaction. Exemplary β-pleated-sheet-binding molecules are listed in SEQ ID NO: 1 to 10.

The term “β-sheet-breaker (BSB)” as used in the present context, describes short peptides, which not only bind to β-pleated-sheet structures of β-amyloid (protein aggregates in Alzheimer's disease) and to amyloid-like structures, but can also block or reverse their abnormal folding.

The term “pathologically-altered prion protein” as used here refers to PrP^{Sc}. PrP^{Sc} can be present both in monomer and/or oligomer form and also in the form of a fibrillary, amyloid aggregate.

Cattle in which proteinase K-resistant PrP^{Sc} is detected post mortem in the brain stem tissue are defined in the present context as “BSE-positive cattle”.

The present invention relates to a method for the detection of pathologically-altered prion proteins (PrP^{Sc}) comprising the following steps:
a) incubation of a specimen together with a solid carrier, wherein the solid carrier is coupled to a β-pleated-sheet-binding molecule;
b) removal of the constituents of the specimen not bound to the β-pleated-sheet-binding molecules; and
c) detection of the pathologically-altered prion proteins (PrP^{Sc}) bound to the β-pleated-sheet-binding molecules.

With the method according to the invention, the monomer and/or oligomer pathologically-altered prion proteins contained in body fluids, cell lysates or body tissues are enriched in such a manner that even the smallest, hitherto non-detectable concentrations of PrP^{Sc} can be demonstrated. As a result, living animals or humans can be classified as infected even shortly after infection with TSE-triggering prions. This was not previously possible, because sensitive tests of this kind were not available and, moreover, the detection could only be carried out post mortem with brain tissue, in which the concentration of PrP^{Sc} is sufficiently high. Furthermore, because of its high sensitivity, the method provides results at a substantially earlier time after infection in body tissues, e.g. brain homogenates, which show results with the existing detection method only when the infection has already advanced to a significant degree.

The specimen to be investigated can be a body fluid, e.g. blood, serum, plasma, cerebrospinal fluid, aqueous humour, lachrymal fluid, urine, saliva, lymph, milk, or a cell lysate, e.g. from leukocytes or from cells from the lymphatic tissue, or a tissue homogenate, e.g. from tissue from the central nervous system, from lymph tissue (e.g. spleen, tonsils, lymph nodes) or other organs.

Before incubation, the specimen may optionally be subjected to a specimen-preparation stage. This may be necessary particularly in the case of tissue specimens. These can be mechanically comminuted after the addition of an appropriate buffer solution, e.g. 50 mM phosphate buffer, pH 7.5, for example, using ultrasound or ribolyser treatments, and then homogenised, in order to convert their constituents into a solution or suspension. To separate the solid constituents in the tissue or cell suspensions obtained from mechanical treatment or the solid constituents present in the body fluids, the specimen can also be subjected to a centrifuging and/or filtration stage.

With or without the specimen preparation described above, the specimen can optionally, either additionally or exclusively, be subjected to a proteinase treatment to achieve a proteolytic breakdown of PrP^{C} before the incubation. This is primarily indicated if the PrP^{Sc} to be detected, e.g. the monomer and/or oligomer form of PrP^{Sc}, is to be detected using antibodies, which can distinguish PrP^{Sc} from PrP^{C} only after proteinase treatment. For this purpose, the specimen material is treated with a protease, e.g. proteinase K. The protease digestion may be carried out under standard conditions for the respective protease or according to the manufacturer's instructions, preferably for 1 hour at 37° C. The enzyme concentration used may be within a range from approximately 10 μg/ml to approximately 1 mg/ml in dependence upon the specimen material, preferably approximately 50 μg/ml enzyme with a protein content of the specimen material of approximately 0.5 to approximately 10 mg/ml.

The solid carriers can be spherical polymers (e.g. sepharose, agarose or latex), plastic surfaces (e.g. microtitre plates), silica-gel-coated glass plates (e.g. for thin-layer chromatography), capillaries or membranes. The spherical polymers can be used as carriers in column chromatography or in a batch process (e.g. magnetic beads). If the polymers are used for column chromatography, they are preferably used in pre-packed, disposable columns. Alongside the solid carriers listed here, any solid carrier is suitable, which can be used for coupling β-pleated-sheet-binding molecules.

The specimen can be incubated together with the solid carrier, e.g. glass plates, micro-titre plates in an enclosed vessel for approximately 5 to approximately 120 minutes at a temperature within the range from approximately 4° C. to approximately 50° C. The incubation is preferably carried out for 1 hour at 37° C. in an incubator shaker with a low rotational frequency (e.g. 80 rpm). By incubating the specimen together with the solid carrier, the PrP^{Sc} contained in the specimen is bound to the β-pleated-sheet-binding molecule immobilised on the solid carrier. If the solid carrier, for example, a spherical polymer, is used in polymer chromatography, the incubation is performed in the column. The incubation period can vary according to the column, in dependence upon the connection of the column to the apparatus and the through-flow rate of the specimen.

The β-pleated-sheet-binding molecules coupled to the solid carrier bind PrP^{Sc} with a substantially greater affinity than PrP^{C} and, according to the invention, are capable of capturing the soluble and the monomer and/or oligomer forms of PrP^{Sc} occurring in body fluids. According to the invention, the β-pleated-sheet-binding molecules are oligopeptides consisting of 3 to approximately 30 amino acids, preferably 4, 5 or 6 amino acids. These peptides can be C-terminal and/or N-terminal modified, e.g. in order to achieve improved solubility. In addition to their property of binding PrP^{Sc}, the β-pleated-sheet-binding molecules can also provide the properties of β-sheet-breakers [BSB]. However, beyond these BSB properties, the β-pleated-sheet-binding molecules according to the present invention provide binding properties (affinity, reversibility of binding) to the PrP^{Sc}, which allow the capture and enrichment of PrP^{Sc} from solutions. BSB peptides, which bind the β-pleated-sheet structures too firmly or in an irreversible manner, thereby preventing elution, are unsuitable as β-pleated-sheet-binding molecules. BSBs, which bind PrP^{Sc} with too low an affinity or in a non-permanent manner (e.g. release of the PrP^{Sc} from the BSB after the breakdown of the β-pleated-sheet structure) are also unsuitable. Particularly preferred β-pleated-sheet-binding molecules are shown in Table 1 and listed as SEQ ID NO: 1 to 10. The β-pleated-sheet-binding molecule can also be a substituted heterocyclic aromatic, advantageously a flavonoid, for example, thioflavin T, baicalin or quercitrin.

The β-pleated-sheet-binding molecule is preferably immobilised on the solid carrier via a covalent bond. Functional groups, such as amino, carboxyl or hydroxyl groups on the β-pleated-sheet-binding molecule are used to achieve the coupling with the carrier. If the β-pleated-sheet-binding molecule is a peptide, the coupling is preferably achieved via the amino group at the N-terminus or the carboxyl group at the C-terminus. If the oligopeptide is a pentapeptide with the sequence KLVFF (SEQ ID NO:2), the coupling is preferably formed via the carboxyl group at the C-terminus, because, with a coupling via the amino group, the peptide would also be fixed at the side chain of the lysine residue, which could lead to steric hindrance of the PrP^{Sc} binding.

Following the incubation of the specimen together with the solid carrier, the constituents of the specimen not bound to the β-pleated-sheet-binding molecule are removed, preferably in a washing stage. A buffered solution with appropriate stringency-increasing additives is used as the washing solution. The pH value of the washing solution is within the neutral range, preferably approximately pH 7.5. By preference, a 50 mM phosphate buffer is used to buffer the solution. Otherwise, any buffer, which can adjust a pH value in the neutral range, is suitable. The stringency-increasing additives can be inorganic salts, e.g. NaCl, detergents, such as SDS, Triton X 100 or Tween 20, or chaotropic reagents, e.g. urea, guanidine hydrochloride or guanidine isothiocyanate. A buffer solution with 1 to 4 M NaCl is advantageously used as the washing solution.

Depending on the surface properties of the carrier material, the PrP^{Sc} bound to the β-pleated-sheet-binding molecule is optionally eluted from the solid carrier (e.g. when using spherical polymers in column chromatography). With other carriers, e.g. membranes or plastic surfaces, the PrP^{Sc} can be detected directly on the solid carrier. However, elution is also possible in this case, if required.

In order to elute the PrP^{Sc} from the β-pleated-sheet-binding molecule and accordingly from the solid carrier, the carrier is rinsed with an extremely small volume of elution solution. To achieve a concentration effect adequate for the sensitivity of the detection system used, the elution volume should be considerably smaller than the volume of the specimen.

A buffered solution containing additives which dissolve the bond between PrP^{Sc} and the catcher molecule is used as the elution solution. The pH value of the elution solution is within the range from approximately pH 6 to approximately pH 8.5, preferably approximately pH 7.5. By preference, 50 mM phosphate buffer is used to buffer the solution. Otherwise any buffer, which can adjust a pH value in the range described above, preferably in the neutral range, is suitable. The additives may, for example, be detergents, e.g. SDS, Triton X 100 or Tween 20, chaotropic reagents, e.g. urea, guanidine hydrochloride or guanidine isothiocyanate, inorganic salts, e.g. NaCl. The elution solution preferably contains detergents, for example, 5% SDS.

Following this, the PrP^{Sc} enriched in the preceding stages can be detected. For this purpose, immunochemical detection methods (e.g. ELISA, Western Blot, immuno-precipitation); biophysical detection methods (e.g. mass spectrometry, fluorescence correlation spectroscopy); biochemical detection methods (e.g. measurement of biochemical parameters, such as relative molar mass, N-terminal or C-terminal amino-acid sequence, association and dissociation constants of binding partners); or biological detection methods (e.g. cytotoxicity assay) can be used.

By preference, the detection is carried out with a method, which allows a rapid detection of PrP^{Sc}. This can be, for example, an immunological detection method, preferably a sandwich-ELISA. The sandwich ELISA is performed using known methods. In this context, the detection antibody e.g. an enzyme (e.g. horse-radish peroxidase), can be marked with a stained compound, a fluorescence stain (e.g. fluorescein), a gold particle or a nucleic acid (e.g. a DNA or RNA oligonucleotide).

In the case of enzyme marking, the intensity of the stain is registered photometrically after conversion of the substrate and is proportional to the quantity of PrP^{Sc} contained in the specimen. If the antibody marking is a stained compound or a fluorescence stain, the intensity of the stain or respectively the fluorescence is measured directly. If the antibody marking is a nucleic acid, the quantity of bound antibody is measured via the absorption of the DNA or RNA label, wherein the signal is amplified using PCR (e.g. real-time PCR).

The present invention also relates to a kit for the detection of PrP^{Sc} in body fluids, cell lysates, tissue homogenates or other fluids. According to the invention, the test kit contains a solid carrier for the enrichment of PrP^{Sc}, an immunological detection system, solubilising, washing and elution buffer concentrates, various controls, an enzyme-marked anti-PrP-antibody and a corresponding substrate and stop solution.

The solid carriers used are preferably affinity-chromatographic materials, e.g. sepharose, which are used in disposable columns, or on plastic surfaces, e.g. microtitre plates, which are coupled with the β-pleated-sheet-binding molecules according to the invention. If the solid carriers are affinity-chromatographic materials with the couplings according to the invention, these may be contained in suspension, in dried form or may already be packed in disposable columns in the test kit.

The immunological detection system is preferably a sandwich ELISA, in which a second solid carrier, e.g. a micro-titre plate is coated with a specific antibody to PrP, preferably with monoclonal anti-PrP-antibodies, in particular, mouse-anti-PrP-antibodies. The solid carriers in the test kit are, in particular, provided in a vacuum-packed manner.

PrP and/or PrP peptides manufactured in a recombinant manner are preferably used as controls. Horse-radish peroxidase is preferably used as the antibody marking.

The methods and test kits according to the invention allow broadly designed investigations with large numbers of specimens, as required in the fields of medicine and agriculture. Automation of the detection method in an appropriately equipped laboratory is possible. By contrast with all previously described methods, the methods according to the invention are also suitable for TSE diagnostics with living animals and humans.

The invention will be explained in greater detail with reference to the following examples:

### EXAMPLE 1

### Isolation of PrP^{Sc} from Brain Homogenate Using Different Peptides in MTP Format

A microtitre plate (MTP) (Nunc-Immuno™ Plate Maxisorp™ Surface, F96 (Nunc, Roskilde, Denmark)), was coated with the peptides listed in Table 1 (see FIG. 2). The coating was performed by incubation with 100 μl peptide solution (10 μg/ml in 0.1 M carbonate buffer pH 9.6) per cavity for 16 h at 4° C. The fluid was vacuumed off and the MTP was washed three times with 300 μl washing buffer (PBS (10 mM phosphate buffer, 0.15 M NaCl, pH 7.2); 0.05% Tween 20)) per cavity. Free binding positions were blocked by incubation with 0.5% casein in washing buffer at room temperature for 1 hour.

After a washing stage (300 μl washing buffer per cavity), the coated MTP was covered with foil and incubated with 100 μl per cavity of a PrP^{Sc}-containing specimen (brain homogenate from a BSE-positive animal, with OD>4.0 in the Platelia®; the positive finding was confirmed by immuno-histological investigation of the brain tissue) for 1 hour at 37° C. Non-bound specimen material was vacuumed off and the MTP was washed three times with 300 μl washing buffer per cavity. The incubation with the detection antibody (Platelia® BSE Detection, Kit, Bio-Rad Laboratories, Hercules, USA) was performed for 1 hour at room temperature according to the manufacturer's instructions. Surplus detection antibody was removed by washing five times with 300 μl washing buffer per cavity. After adding the substrate solution (Tetramethylbenzidine [TMB], Platelia® BSE Detection Kit, Bio-Rad Laboratories, Hercules, USA), the stain development was stopped after 30 minutes by the addition of 1M H₂SO₄ and the intensity of stain was registered by extinction measurement at 450 nm (reference 620 nm).

The measured extinction is proportional to the quantity of PrP^{Sc} bound to the peptides and therefore provides a measure for the efficiency of the catcher molecule. The relative binding efficiencies of the peptides tested are summarized in Table 1, the signal from the best β-pleated-sheet-binding molecule (peptide 2) being set at 100%.

TABLE 1
Comparison of PrP^{Sc} binding efficiency of various peptides

| | | |
|---|---|---|
| No. | Sequence | Efficiency |

| | | | |
|---|---|---|---|
| Peptide 1 | Arg-Val-Val-Ile-Ala | 54.7 | SEQ ID NO: 1 |
| Peptide 2 | Lys-Leu-Val-Phe-Phe | 100.0 | SEQ ID NO: 2 |
| Peptide 3 | Leu-Pro-Phe-Phe-Asp | 46.6 | SEQ ID NO: 3 |
| Peptide 4 | Propionyl-Ile-Ile-Gly-Leu | 55.1 | SEQ ID NO: 4 |
| Peptide 5 | Propionyl-Arg-Ile-Ile-Gly-Leu | 58.1 | SEQ ID NO: 5 |
| Peptide 6 | Gly-Val-Val-Ile-Ala | 64.5 | SEQ ID NO: 6 |
| Peptide 7 | Propionyl-DArg-DArg-DA1a-DPhe-DPhe-DVal-amide | 76.5 | SEQ ID NO: 7 |

### EXAMPLE 2

### Elution of the PrP^{Sc} Bound to KLVFF in MTP Format

The bond between the PrP^{Sc} and the catcher molecules is very strong, and comparatively drastic conditions are necessary in order to elute the PrP^{Sc} from the solid carrier. The elution conditions were also tested using the MTP format.

As with Example 1, an MTP was coated with peptide 2 (KLVFF) and charged with brain homogenate containing PrP^{Sc} (OD in Platelia®>3.0). After washing three times with 300 μl washing buffer (PBS; 0.05% Tween 20) per cavity, 100 μl of the potential elution buffer (see Table 2) were added and incubated for 5 minutes at room temperature. Following this, the fluid was removed and the eluted quantity of PrP^{Sc} in the elution buffer and the remaining quantity of PrP^{Sc} on the MTP was measured using the Platelia® BSE Detection Kit (Bio-Rad Laboratories, Hercules, USA). Comparison of the efficiency of elution (Table 2) showed that only a detergent-containing buffer (with 5% SDS) was suitable for the complete elution of PrP^{Sc} from the catcher molecule. In the presence of chaotropic reagents (e.g. 6M urea) PrP^{Sc} was only partially eluted. In elution buffers with a low pH value (e.g. pH 3) or respectively with a high ionic strength (e.g. 2M NaCl), PrP^{Sc} remained almost completely bound to the catcher molecule.

TABLE 2
comparison of various elution conditions

| | | | Elution |
|---|---|---|---|
| | Eluent | Buffer composition | efficiency |
| A | 2 M NaCl | 20 mM phosphate buffer, 2 M NaCl, pH 7.4 | +/− |
| B | pH 3 | 100 mM glycin/HCl buffer, pH 3.0 | +/− |
| C | 6 M urea | 20 mM phosphate buffer, 6 M urea, pH 7.4 | + |
| D | 5% SDS | 20 mM phosphate buffer, 5% SDS, pH 7.4 | +++ |

### EXAMPLE 3

### Covalent Coupling of the Peptide KLVFF to EAH-Sepharose

In the case of a coupling via amino groups, the peptide would be fixed both to the N-terminus and also to the side chain (Lys), which could disturb the three-dimensional structure. For this reason, the specific binding of the β-pleated-sheet-binding molecule KLVFF to the solid carrier was achieved via the carboxyl group at the C-terminus of the peptide. EAH-Sepharose 4B (Amersham Pharmacia Biotech, Uppsala, Sweden) was used as a carrier material.

In order to prepare the coupling reaction, the EAH-Sepharose was washed with 0.5M NaCl, and any surplus fluid was completely removed. The ligand, the pentapeptide with the sequence KLVFF, was dissolved in H₂O to a final concentration of 5 mg/ml and the pH was adjusted to 4.5 with HCl. The gel was re-suspended in the ligand solution (1 part gel+2 parts ligand solution), and EDC (N-ethyl-N′-(3-dimethylaminopropyl)-carbodiimide) was added in a final concentration of 0.1M. The coupling reaction was performed at room temperature over 24 hours with gentle rotation. Following this, the supernatant was completely removed from the gel sediment. According to the manufacturer's recommendations, any free binding positions present were blocked with 1M acetic acid in the presence of 0.1M EDC. The KLVFF-charged gel was placed in chromatography columns (bed volume 1 ml) and washed at least three times alternately in each case with 3×2 ml buffer A (0.1M Na-acetate, 0.5M NaCl, pH 4) and 3×2 ml buffer B (0.1M tris/HCl, 0.5M NaCl, pH 8) and finally with 10×2 ml H₂O.

### EXAMPLE 4

### Isolation of PrP^{Sc} from Brain Homogenate by Means of KLVFF-Sepharose

To demonstrate the suitability of the KLVFF-sepharose as a β-pleated-sheet-binding molecule, PrP^{Sc} from brain homogenate of BSE-positive cattle was bound to the column material and then eluted again. The brain material was prepared with the BSE Purification Kit (Bio-Rad Laboratories, Hercules, USA) according to the manufacturer's instructions. The specimen material was also dissolved according to the manufacturer's instructions in specimen dilution buffer R6 (Platelia® BSE Detection Kit, Bio-Rad Laboratories, Hercules, USA) (OD in Platelia® 6.0).

A drip column was prepared with 1 ml KLVFF-sepharose, as indicated in Example 3, filled and equilibrated with PBS at room temperature. After the application of the specimen (250 μl), the column was washed with 2 ml PBS, and the run-off was collected in fractions. Bound PrP^{Sc} was then eluted in fractions with 1.5 ml 5% SDS in PBS. The quantity of PrP^{Sc} obtained in the individual fractions was measured immunologically using the Platelia® BSE Detection Kit.

FIG. 3 shows the elution profile of this experiment and records the capability of the KLVFF-sepharose for reversible binding of PrP^{Sc}, and therefore the suitability of this matrix for selective enrichment of PrP^{Sc} from large specimen volumes. In this context, the PrP^{Sc} contained in the run-off is attributable to an overloading of the column capacity, because the brain specimen used here had a very high PrP^{Sc} content (OD in Platelia® 6.0). The signal obtained in the eluate is reduced by the disturbing influence in the ELISA of the SDS in the elution buffer.

### EXAMPLE 5

### Isolation of PrP^{Sc} from Body Fluids by Means of KLVFF in MTP-Format (Priontype In-Vivo BSE-Test)

An MTP (Nunc-Immuno™ Plate Maxisorp™ Surface, F96 (Nunc, Roskilde, Denmark)) was coated with the peptide KLVFF (FIG. 2). The coating was provided by incubation with 100 μl peptide solution (10 μg/ml in 0.1M carbonate buffer pH 9.6) per cavity for 16 hours at 4° C. Following this, the fluid was vacuumed off and the MTP was washed three times with 300 μl washing buffer (PBS; 0.05% Tween 20; pH 7.2) per cavity. Free binding positions were blocked by incubation with 0.5% casein in washing buffer at room temperature for 1 hour. After a washing stage (three times 300 μl washing buffer per cavity), the coated MTP was covered with foil and incubated with 100 μl per cavity of the PrP^{Sc}-containing specimen for 1 hour at room temperature.

In this experiment, aqueous-humour specimens from 9 BSE-positive and 5 BSE-negative cattle and cerebrospinal fluid specimens from 6 BSE-positive and 13 BSE-negative cattle were investigated. All the specimens had previously been prepared with the BSE Purification Kit (Bio-Rad Laboratories, Hercules, USA). Non-bound specimen material was vacuumed off and the MTP was washed three times with 300 μl washing buffer per cavity. Incubation with the detection antibody (5 μg/ml V5B2 in washing buffer, r-Biopharm, Darmstadt) was performed for 1 hour at room temperature. Following this, the plate was again washed three times with 300 μl washing buffer, and incubated for 1 hour at room temperature with a goat-anti-mouse IgG-peroxidase conjugate (1:20000 in washing buffer, Jackson, USA). Surplus conjugate was removed by washing five times with 300 μl washing buffer per cavity. After adding the substrate solution (TMB), the stain development was stopped after 15 minutes by the addition of 1M H₂SO₄, and the stain intensity was registered by extinction measurement at 450 nm (reference 620 nm). The measured extinction is proportional to the quantity of PrP^{Sc} bound to the peptides. As can be seen from FIG. 4, the values obtained with the BSE-positive animals are significantly (t-test) raised by comparison with BSE-negative animals. The differences between positive and negative specimens in the aqueous-humour specimens are substantially more pronounced than in the cerebrospinal fluid. This is explained by the PrP^{Sc} concentration occurring in the corresponding body fluids. In view of these results, aqueous humour is preferable to cerebrospinal fluid as a specimen material for the detection of PrP^{Sc} in the body fluids of living animals.

1. (canceled)

2. (canceled)

3. (canceled)

4. (canceled)

5. (canceled)

6. (canceled)

7. (canceled)

8. (canceled)

9. (canceled)

10. A method for the detection of pathologically-altered prion proteins (PrP_{Sc}), comprising the following steps:
a) incubating a specimen together with a solid carrier, wherein the solid carrier is coupled with a β-pleated-sheet-binding molecule, wherein the β-pleated-sheet-binding molecule is an oligopeptide, selected from the group consisting of oligopeptides of the amino-acid sequences according to SEQ ID NO: 1 to 10, or a substituted heterocyclic aromatic, selected from the group consisting of thioflavin T, baicalin and quercitrin;
b) removing the constituents of the specimen not bound to the β-pleated-sheet-binding molecules; and
c) detecting the pathologically-altered prion proteins (PrP^{Sc}) of the constituents of the specimen bound to the β-pleated-sheet-binding molecules.

11. A method according to claim 10, wherein the specimen is subjected to a proteinase treatment before step a).

12. A method according to claim 10, wherein the solid carrier is a spherical polymer, a plastic surface, silica-gel-coated glass slide, capillary or membrane.

13. A method according to claim 10, wherein the detection is performed by means of an immunological detection method.

14. A method according to claim 11, wherein the specimen is subjected to a proteinase treatment before step a).

15. A method according to claim 11, wherein the solid carrier is a spherical polymer, a plastic surface, silica-gel-coated glass slide, capillary or membrane.

16. A method according to claim 11, wherein the detection is performed by means of an immunological detection method.

17. A kit for the detection of pathologically-altered prion proteins (PrP^{Sc}), comprising at least one solid carrier coupled with β-pleated-sheet-binding molecules, washing solutions, elution solutions and a detection system.

18. A kit according to claim 17, wherein the detection system is an immunological detection system and comprises a second solid carrier, which is coated with an anti-PrP-antibody, an enzyme-marked second antibody, substrate solutions and stop solutions.

19. A kit according to claim 17, wherein one solid carrier is packed in a disposable column, and the second solid carrier is a microtitre plate.

20. A kit according to claim 18, wherein one solid carrier is packed in a disposable column, and the second solid carrier is a microtitre plate.
